(19) United States
(12) Patent Application Publication
(10) Pub. No.: US 2003/0157067 A1
(43) Pub. Date: Aug. 21, 2003

(54) Formulation and method for preventing antibiotics-induced diaper rash

(76) Inventor: Jennifer F. Bartels, Hammond, IA (US)

Correspondence Address: JENNIFER F. BARTELS, 601 N. HAZEL ST., HAMMOND, IA, 70401, US

(21) Appl. No.: 10/369,367

(22) Filed: Feb. 18, 2003

Related U.S. Application Data
(60) Provisional application No. 60/358,332, filed on Feb. 19, 2002.

(51) Int. Cl. A61K 45/00; A61K 31/70

(52) U.S. Cl. 424/93.3; 514/23

(57) ABSTRACT

A formulation and method for the prevention of antibiotics induced diaper rash comprising a) live active cultures selected from the group consisting of lactobacillus bulgaricus, streptococcus thermophilus, lactobacillus acidophilus, bifidus, and mixtures thereof; b) a pharmaceutically acceptable carrier; c) sugars or syrups selected from the group consisting of glucose, dextrose, fructose, sucrose, and derivatives and mixtures thereof, and d) flavorings. The pharmaceutically acceptable carrier is selected from the group consisting of purified water, soy milk, infant formula, juices, and mixtures thereof. The formulation might further comprise preservatives, diluents, fillers, starches and coloring. In its preferred embodiment, the formulation would be placed, poured or mixed into a dropper dispenser bottle for infant dosage.

### BACKGROUND OF THE INVENTION

The present invention relates to a convenient liquid formulation for children and infants who are too young to chew probiotic tablets or consume probiotic drink mixes or persons who otherwise are unable to ingest other forms of probiotics due to age, illness, or disability. It has long been known that antibiotics dosages often cause diarrhea. The resulting diaper rash which invariably accompanies this diarrhea due to the intestinal flora imbalance caused by the antibiotics is a cause of great discomfort for both infants, patients, and caregivers.

The present invention focuses on the diaper rash which results from intestinal flora imbalances that occur during antibiotic dosages.

It is difficult to say that there is prior art relating to the diaper rash component of intestinal imbalances, since none of the many probiotics formulations found ever mentioned the problem of diaper rash.

However, other prior art was found relating to probiotics formulations. U.S. Pat. No. 6,468,525 issued to Watson does not address the issue of diaper rash and does not go about in helping solve this uncomfortable condition in the practical and safe manner in which the present invention seeks to. The present invention seeks only to mimic in the infant's intestines the probiotic effect of ingesting a single or double age appropriate serving of live active cultures yogurt (LAC certified) for the 10 day duration of most antibiotics dosages.

Moreover, the prior art seeks to be used as a food supplement rather than a pharmaceutical support to alleviate a transient condition caused by medication.

Other prior art, U.S. Pat. No. 5,906,982 issued to Prieto seeks to invent a probiotic formula. This prior art does not help the infant or caregiver as it relates to antibiotics induced diaper rash if the infant is entirely breastfed (i.e., taking no formula, bottles, or other food types.) The present invention is superior also because even if the infant is formula fed, a disruption in formula type other than what the infant is used to could cause more intestinal upset than the probiotic is able to alleviate. A probiotic supplement other than a change in formula would provide a smooth, nonconflicting supplement which can only enhance the infant's comfort level as well as the peace of mind of the caregiver. The prior art does not even mention the problem of diaper rash.

In spirit, the prior art has a main goal of becoming a food or food supplement with other nutritional value rather than a prevention for a transient condition such as diaper rash.

Other prior art references indude U.S. Pat. No. 5,902,578 issued to Halpin-Dohnalek which purports a method and formula for the prevention of diarrhea. This prior art mentions mixing live cultures into a liquid, but said liquid again purports to be a nutritional product or formula rather than a pharmaceutical support mechanism for antibiotics dosages. Again, the transient but recurring problem of diaper rash is never mentioned.

Tens to hundreds of patented compositions for treating diaper rash exits. The present invention seeks to prevent rather than treat antibiotics induced diaper rashes. It would stand to reason that if possible, as it benefits infants and caregivers greatly, a prevention would be superior to a treatment in any reasonable assessment.

Consequently, a need has been felt for providing a convenient, safe, and beneficial treatment for the transient problem of infant diaper rash as it relates to antibiotics dosages.

The novelty of the present invention is that it seeks simply to provide the live active cultures in an age appropriate serving of yogurt in a convenient dropper form. An ill infant or one that is either breastfeeding or not yet eating table foods cannot benefit from many of the prior art referenced supplements.

Moreover, the present invention is unique and innovative due to its formulation, method, and difference in approach to the prior art in preventing antibiotics induced diaper rash.

### SUMMARY OF THE INVENTION

It is therefore an object of the present invention to provide an innovative and effective formulation to prevent antibiotics induced diarrhea.

There are other intestinal disturbances which may be helped by the present invention, including diaper rash resulting from teething (acidic saliva making its way to the intestines), an acidic diet (too much juice), and bacterial infections which disrupt the intestinal flora, including rotavirus.

It is a feature of the present invention to provide a novel use for the formulation in the form of a dropper dispenser bottle which is pre-mixed, possibly but not necessarily refrigerated after mnxing, and ready to dispense to the infant. Agitation may be necessary to evenly disperse the active cultures within the formulation.

Advantages of the present invention are that it is easily given to the infant, the technology exists to create this mixture cost effectively, and the active cultures and other ingredients have been proven to be safe over years of use in pediatric, nutritional, and home environments and applications.

The formulation is effective by itself or in combination with conventional treatments such as barrier creams, diaper additives, or other topical compositions.

### DESCRIPTION OF THE PREFERRED EMBODIMENTS

It has now been discovered, a formulation that comprises cultures normally found in yogurt selected from the group consisting of lactobacillus bulgaricus, streptococcus thermophilus, lactobacillus acidophilus, bifidus, and mixtures thereof. These live cultures would function in a preparation of sugars or syrups selected from the group consisting of sucrose, dextrose, fructose, glucose, and derivatives and mixtures thereof; along with a pharmaceutically acceptable carrier, and flavorings. The pharmaceutically acceptable carrier would be selected from the group consisting of purified water, soy milk, infant formula, juices, and mixtures thereof. The formulation might also comprise fillers, starches, preservatives, and mixtures thereof.

The active cultures would provide some means of prevention of antibiotics induced diaper rash by helping replace friendly intestinal flora.

### EXAMPLE 1

In its preferred embodiment, the formulation is made of the following ingredients:

a. 1-50% lactobacillus bulgaricus (1 million-50 billion)

b. 1-50% streptococcus thermophilus (1 million-50 billion)

c. 1-50% lactobacillus acidophilus (1 million-50 billion)

d. 1-50% bifidus (1 million-50 billion)

e. 5-95% pharmaceutically acceptable carrier

f. 5-50% sugars or syrups

g. 1-40% flavoring

wherein said pharmaceutically acceptable carrier is selected from the group consisting of purified water, soy milk, infant formula, juices, and mixtures thereof. Sugars or syrups are selected from the group consisting of sucrose, dextrose, fructose, glucose, and derivatives and mixtures thereof.

It is also currently envisioned that for the formulation to have adequate shelf life and viscosity that other pharmaceutically acceptable preservatives, diluents, fillers, starches and carriers may be included. Also the formulation may include the addition of coloring (food grade) to assist in visualizing the dosage given.

Flavoring choices might include: strawberry, chocolate, vanilla, blueberry cheesecake, lemon meringue pie, rasberry tart, or banana cream pie.

### EXAMPLE 2

In a particularly preferred embodiment, the formulation is made of the following ingredients, which would be poured into a 20 ml bottle which will dose a 15-25 pound infant twice a day for a 10 day duration of antibiotics dosage:

a. dry mix or liquid suspension of 50 billion organisms selected from the group consisting of lactobacillus bulgaricus, streptococcus thermophilus, lactobacillus acidophilus, bifidus, and mixtures thereof, most closely resembling those found in live active cultures yogurt (LAC certified) pro rated by per gram composition (100 million live organisms per gram of LAC yogurt.)

b. purified water to complete 20 ml of liquid when combined with organism mixture

c. 5 ml high fructose corn syrup

d. 0.8 ml banana cream pie flavoring

It is also currently envisioned that for the formulation to have adequate shelf life and viscosity that other pharmaceutically acceptable preservatives, diluents, fillers, starches and carriers may be included. Also the formulation may include the addition of coloring (food grade) to assist in visualizing the dosage given.

The foregoing descriptions are included to illustrate the operation of the preferred embodiment and is not meant to limit the scope of the invention. From the foregoing description, many variations will be apparent to those skilled in the art that would yet be encompassed by the spirit and scope of the invention.

1. A formulation for preventing antibiotics induced diaper rash comprising a) live active cultures selected from the group consisting of lactobacillus bulgaricus, streptococcus thermophilus, lactobacillus acidophilus, bifidus, and mixtures thereof; b) a pharmaceutically acceptable carrier; c) sugars or syrups selected from the group consisting of glucose, dextrose, fructose, sucrose, and derivatives and mixtures thereof; and d) flavorings.

2. The formulation of claim 1, wherein said pharmaceutically acceptable carrier is selected from the group consisting of purified water, soy milk, infant formula, juices, and mixtures thereof.

3. The formulation of claim 1, wherein the formulation further comprises preservatives, diluents, fillers, starches and coloring.

4. A formulation for preventing antibiotics induced diaper rash comprising:
a. 1-50% lactobacillus bulgaricus (1 million-50 billion)
b. 1-50% streptococcus thermophilus (1 million-50 billion)
c. 1-50% lactobacillus acidophilus (1 million-50 billion)
d. 1-50% bifidus (1 million-50 billion)
e. 5-95% pharmaceutically acceptable carrier
f. 5-50% sugars or syrups
g. 1-40% flavorings

4. The formulation of claim 4, placed, mixed or poured into a dropper dispenser bottle for infant dosage.

6. A method for preventing antibiotics induced diaper rash in a patient, comprising dispensing formulation in the patient's mouth with a dispensing dropper a formulation comprising: a) live active cultures selected from the group consisting of lactobacillus bulgaricus, streptococcus thermophilus, lactobacillus acidophilus, bifidus, and mixtures thereof; b) a pharmaceutically acceptable carrier; c) sugars or syrups selected from the group consisting of glucose, dextrose, fructose, sucrose, and derivatives and mixtures thereof; and d) flavorings.

7. The method of claim 6, wherein said pharmaceutically acceptable carrier is selected from the following group consisting of purified water, soy milk, infant formula, juices, and mixtures thereof.

8. The method of claim 6, wherein the formulation further comprises preservatives, diluents, fillers, starches and coloring.
